(19) United States
(12) Patent Application Publication
(10) Pub. No.: US 2024/0285600 A1
(43) Pub. Date: Aug. 29, 2024

(54) NOVEL MKRN1 INHIBITOR AND PHARMACEUTICAL COMPOSITION FOR PREVENTING OR TREATING OBESITY OR FATTY LIVER COMPRISING THE SAME

(71) Applicant: UIF (University Industry Foundation), Yosei University, Seoul (KR)

(72) Inventors: Jaewhan Song, Seoul (KR); Seungyeon Kim, Seoul (KR); Hyunjin Rho, Seoul (KR)

(21) Appl. No.: 18/400,602

(22) Filed: Dec. 29, 2023

(30) Foreign Application Priority Data
Feb. 22, 2023 (KR) 10-2023-0023711

(51) Int. Cl. A61K 31/445; A61P 1/16; A61P 3/04

(52) U.S. Cl. CPC A61K 31/445; A61P 1/16; A61P 3/04

(57) ABSTRACT

The present invention relates to a novel MKRN1 inhibitor and a medicinal use thereof. Specifically, the present invention relates to a method of treating obesity, diabetes, fatty liver disease, and/or steatohepatitis by administering ebastine to a subject in need of treatment.

### CROSS-REFERENCE TO RELATED APPLICATION

This application claims priority to and the benefit of Korean Patent Application No. 10-2023-0023711, filed on Feb. 22, 2024, the disclosure of which is incorporated herein by reference in its entirety.

### BACKGROUND

### 1. Field of the Invention

The present invention relates to a novel MKRN1 inhibitor, and more particularly, to a pharmaceutical composition for preventing or treating obesity and/or fatty liver disease, which includes ebastine as an active ingredient.

### 2. Discussion of Related Art

Obesity is a phenomenon in which excess energy is accumulated as fat in the body due to an imbalance in the metabolic process caused by endocrine factors, genetic factors, eating habits, social and environmental factors, and the like. Obesity causes visible symptoms such as shortness of breath, arthralgia, and the like, but it is more serious in that it causes various complications. Complications caused by obesity include cardiovascular disease, hypertension, sleep apnea, dyslipidemia, and the like. Therefore, the World Health Organization regards obesity as a disease that must be treated rather than as a simple risk factor that harms health.

Meanwhile, fatty liver is the most common liver disease and generally refers to the excessive accumulation of fat in the liver. That is, fatty liver refers to a condition in which the content of neutral lipids is 5% or more. Fatty liver disease may be classified into non-alcoholic fatty liver disease (NAFLD), which is mainly caused by metabolic risk factors (metabolic syndrome) such as diabetes, obesity, hyperlipidemia, and the like, and alcoholic fatty liver disease caused by alcohol consumption, that is, excessive drinking. Non-alcoholic fatty liver disease is caused by causes other than fatty liver caused by alcohol, and its symptoms include inflammation, liver fibrosis, hepatic cirrhosis, and the like. Non-alcoholic fatty liver disease may be divided into simple steatosis and non-alcoholic steatohepatitis (NASH). NASH is a condition in which fat deposition is accompanied by liver cell damage and inflammation, which may lead to liver fibrosis, hepatic cirrhosis, and hepatocellular carcinoma.

Patients suffering from metabolic diseases such as obesity, diabetes, and the like are receiving a lot of attention due to the high incidence of fatty liver disease, but there are no effective therapies or therapeutic agents. Obesity and NASH inhibitor substances being developed by multinational companies are failing in FDA approval due to the lack of sustained drug activity. For example, by 2020, obeticholic acid (OCA), which is a farnesoid X receptor (FXR) agonist, elafibranor, which is a peroxisome proliferator-activated receptor (PPAR) delta agonist, aramchol (arachidyl amido cholanoic acid), which is a lipogenesis inhibitor, saroglitazar, which is a PPAR gamma agonist, cenicriviroc, which is a chemokine receptor type 3 (CCR3) and CCR5 receptor agonist, liraglutide and semaglutide, which are GLP-1 agonists, and the like failed in NASH clinical trials.

Sibutramine (Abbott), which is an obesity treatment, was found to increase the risk of cardiovascular disease, and lorcaserin (Arena Pharmaceuticals; product name: Belviq) was found to increase the risk of developing cancer, and their approval by the U.S. Food and Drug Administration (FDA) has been revoked.

Failure to develop drugs to control metabolic diseases has occurred due to complex reasons over a long period of time and is due to the difficulty in producing an overall effect on metabolic diseases having various symptoms and the development of resistance to the protein mechanism to be targeted. For this reason, there is a need to develop new drugs or combination drugs that act complexly beyond the previously known target proteins.

### SUMMARY OF THE INVENTION

In previous research, the present inventors have found that metabolic diseases such as obesity, diabetes, fatty liver disease, and the like may be ameliorated by suppressing the expression or activity of makorin ring finger protein 1 (MKRN1) (Nature Communications volume 9, Article number: 3404 (2018)), and confirmed through additional research that ebastine inhibits the expression of MKRN1.

Therefore, the present invention is directed to providing a composition for inhibiting MKRN1, which includes ebastine, and a pharmaceutical composition for preventing or treating obesity, diabetes and/or fatty liver, which includes ebastine. According to an aspect of the present invention, there is provided a composition for inhibiting MKRN1, which includes ebastine or a pharmaceutically acceptable salt thereof as an active ingredient.

MKRN1 is an E3 ligase that promotes the degradation of AMP-activated kinase (AMPK), which activates catabolism during cellular metabolism. Because the E3 ligase is involved in the reaction of various proteins, it is difficult to screen for substances that directly target the MKRN1 protein. Accordingly, based on the fact that MKRN1 decomposes AMPK, the present inventors checked whether a substance that activates AMPK can inhibit MKRN1, and found ebastine. As can be seen from the results in FIG. 8, not all AMPK activators can inhibit MKRN1.

Ebastine is an H1 antihistamine used as an allergy treatment, and is known to increase the sensitivity of cancer cells to chemotherapeutic anticancer agents and reverse multidrug resistance in breast cancer, but nothing is known about its relationship with MKRN1. Ebastine has a structure of the following Chemical Formula 1.

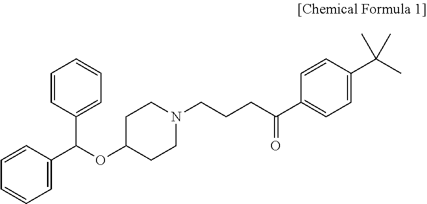 [SMILES: CC(C)(C)c1ccc(C(=O)CCCN2CCC(OC(c3ccccc3)c3ccccc3)CC2)cc1]

As used in this specification, the term “pharmaceutically acceptable salt” refers to an acid addition salt formed by a pharmaceutically acceptable free acid. The acid addition salt is obtained from inorganic acids such as hydrochloric acid, nitric acid, phosphoric acid, sulfuric acid, hydrobromic acid, hydroiodic acid, nitrous acid, or phosphorous acid; and non-toxic organic acids such as aliphatic mono- and dicarboxylates, phenyl-substituted alkanoates, hydroxyalkanoates and alkanedioates, aromatic acids, aliphatic and aromatic sulfonic acids. These pharmaceutically non-toxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, or mandelate, but the present invention is not limited thereto.

The acid addition salt may be prepared by a conventional method, for example, by dissolving the compound represented by Chemical Formula 1 in an excess amount of an aqueous acid solution, and precipitating such a salt in a water-miscible organic solvent, such as methanol, ethanol, acetone, or acetonitrile. Also, the acid addition salt may be prepared by evaporating the solvent or excess acid from such a mixture, followed by drying the mixture or by suction-filtering the precipitated salt.

Also, a pharmaceutically acceptable metal salt may be prepared using a base. An alkali metal or alkaline earth metal salt may be obtained, for example, by dissolving the compound in an excess amount of an alkali metal hydroxide or alkaline earth metal hydroxide solution, filtering the non-dissolved compound salt, and evaporating and drying the filtrate. In this case, it is pharmaceutically suitable to prepare a sodium, potassium, or calcium salt as a metal salt. A corresponding silver salt may be obtained by reacting an alkali metal or alkaline earth metal salt with a suitable silver salt (e.g., silver nitrate).

According to one embodiment, AMPK is highly activated in the liver and adipose tissue of MKRN1 knockout mice (FIG. 1). It is known that the activation of AMPK promotes intracellular catabolism and consequently suppresses the accumulation of fat and the like. Likewise, the activation of AMPK in MKRN1 knockout mice suppressed fat accumulation (FIG. 2), and blood sugar dropped quickly even when glucose was injected into the mice, and blood sugar tended to recover quickly even when insulin was injected into the mice, thereby showing resistance to diabetes (FIG. 3).

In addition, when MKRN1 was knocked down in the liver in order to confirm whether the inhibition of MKRN1 is associated with obesity and diabetes, it was observed that the fat accumulation in the liver was greatly reduced (FIG. 4).

Based on these results, it was confirmed that the inhibition of MKRN1 may ameliorate metabolic diseases, and ebastine was discovered as an MKRN1 inhibitor. Therefore, ebastine may be usefully used for the prevention or treatment of metabolic diseases such as obesity, diabetes, hyperlipidemia, hyperglycemia, or fatty liver disease. The metabolic disease refers to a collection of abnormal conditions such as increased body fat, increased blood pressure, increased blood glucose, blood lipid abnormalities, and the like. Therefore, the metabolic disease is not a single disease, but a comprehensive disease that is caused by a combination of genetic predispositions and environmental factors such as eating habits and the like.

Accordingly, according to another aspect of the present invention, there is provided a pharmaceutical composition for preventing or treating obesity or diabetes, which includes ebastine or a pharmaceutically acceptable salt thereof as an active ingredient.

The term “obesity” refers to a state in which excess energy causes an increase in the quantity and number of fat cells in the body, resulting in excessive accumulation of adipose tissue. When obesity persists, abnormalities may occur in the in vivo metabolic process, and one or more of the following symptoms may appear along with the obesity condition: insulin resistance, type 2 diabetes, hyperlipidemia, fatty liver disease, or inflammation.

Diabetes refers to a type of metabolic disease in which an amount of insulin secretion is insufficient or normal function is not achieved, and is a disease characterized by hyperglycemia, which increases the concentration of glucose in the blood. As shown in FIG. 3A, MKRN1^{-/-} mice have a lower level of increase in blood glucose and a rapid decrease in blood glucose, compared to wild-type mice, which are the control group, even when glucose is administered to the mice, which indicates that blood glucose may be controlled by inhibiting the expression or activity of MKRN1. Therefore, ebastine may be used for anti-diabetic purposes.

As used in the present invention, the term “prevention” refers to all actions that inhibit the progression of or delay the onset of obesity and/or diabetes by administering the pharmaceutical composition according to the present invention.

As used in the present invention, the term “treatment” refers to all actions in which obesity and/or diabetes is improved or beneficially changed by administration of the pharmaceutical composition according to the present invention.

In the present invention, the term “active ingredient” or “pharmaceutically effective amount” refers to any component or amount of the composition used in a process of carrying out the invention provided herein that is sufficient to alleviate, inhibit the progression of, or prevent a disease, a disorder, or a condition, or one or more symptoms thereof.

The pharmaceutical composition of the present invention may further include appropriate carriers, excipients, and diluents commonly used in the preparation of drugs.

The pharmaceutical composition according to the present invention may be formulated and used in the form of oral formulations such as powders, granules, tablets, capsules, suspensions, emulsions, syrups, aerosols, and the like, or in the form of external preparations, suppositories, and sterile injection solutions using conventional methods. The carriers, excipients and diluents that may be included in the composition of the present invention include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

When formulated, the pharmaceutical composition is prepared using commonly used diluents or excipients such as fillers, extenders, binders, wetting agents, disintegrants, surfactants, and the like. Solid preparations for oral administration include tablets, pills, powders, granules, capsules, and the like. These solid preparations are prepared by mixing one or more excipients, for example, starch, calcium carbonate, sucrose, lactose, or gelatin with the composition of the present invention. In addition to simple excipients, lubricants such as magnesium stearate and talc are also used. Liquid preparations for oral use include suspensions, oral liquids, emulsions, syrups, and the like. In addition to the commonly used simple diluents such as water and liquid paraffin, the liquid preparations may include various excipients such as wetting agents, sweeteners, fragrances, preservatives, and the like. Preparations for parenteral administration include sterile aqueous solutions, non-aqueous solvents, suspensions, emulsions, lyophilized preparations, and suppositories. Propylene glycol, polyethylene glycol, vegetable oils such as olive oil, injectable esters such as ethyl oleate, and the like may be used as non-aqueous solvents and suspensions. As a base for suppositories, Witepsol, Macrogol, Tween 61, cacao butter, laurin butter, glycerol gelatin, and the like may be used.

The composition of the present invention may be administered via either an oral or parenteral route of administration, and may also be administered systemically or locally, more preferably administered systemically, and most preferably administered intraperitoneally.

The preferred dosage of the composition of the present invention varies depending on the condition and weight of a patient, the severity of a disease, the type of drug, a route and period of administration, but may be appropriately selected by those skilled in the art. The composition may be administered once a day, or may also be administered in divided doses, and the above dosage is not intended to limit the scope of the present invention in any way.

According to still another aspect of the present invention, there is provided a pharmaceutical composition for preventing or treating fatty liver disease, which includes ebastine or a pharmaceutically acceptable salt thereof as an active ingredient.

As used in the present invention, the term “prevention” refers to all actions that inhibit the progression of or delay the onset of fatty liver disease by administering the pharmaceutical composition according to the present invention.

As used in the present invention, the term “treatment” refers to all actions in which fatty liver disease is improved or beneficially changed by administration of the pharmaceutical composition according to the present invention.

The present inventors confirmed that the expression of MKRN1 significantly increased in the liver of patients with non-alcoholic fatty liver disease (NAFLD) and non-alcoholic steatohepatitis (NASH), while the expression of AMPK was suppressed (FIG. 5).

Then, when NASH was induced by feeding MKRN1^{-/-} mice a high-fat/high-fructose diet, it was confirmed that the liver of the MKRN1^{-/-} mice maintained a normal shape and that fat accumulation and liver fibrosis were suppressed (FIGS. 6 and 7).

Also, it was confirmed that the same results as those for MKRN1-mice were obtained when wild-type mice were fed a high-fat/high-fructose diet and ebastine together (FIGS. 13 and 14), and steatohepatitis was ameliorated when ebastine was administered to mice in which steatohepatitis was induced (FIG. 15). Therefore, ebastine may be usefully used in the treatment of fatty liver disease.

In this specification, non-alcoholic fatty liver disease (NAFLD) refers to a condition in which fat is accumulated in liver cells even when one does not drink alcohol at all or drinks only a small amount of alcohol. Specifically, it includes various types of liver diseases ranging from simple fatty liver disease without inflammation to non-alcoholic steatohepatitis (NASH) and liver cirrhosis.

In the present invention, fatty liver disease may be a non-alcoholic fatty liver disease or non-alcoholic steatohepatitis.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows the results of confirming the levels of AMPK and phospho-AMPK in adipose and liver tissues after makorin ring finger protein 1 (MKRN1) knockout mice are fed a high-fat diet;

FIG. 2 shows the results of measuring the body weights of wild-type and MKRN1-4-mice after the mice are fed a normal or high-fat diet:

FIG. 3 shows the results of measuring changes in blood glucose by feeding the wild-type and MKRN14 mice an HFD for 16 weeks, fasting the mice, and injecting glucose (A) or insulin (B):

FIG. 4 shows the results of observing liver adipocytes after an adenovirus expressing MKRN1 shRNA is injected into the wild-type mice:

FIG. 5A shows the results of staining the liver tissue of patients with non-alcoholic fatty liver disease (NAFLD) and non-alcoholic steatohepatitis (NASH) with hematoxylin & eosin (H&E), and MKRN1 and pAMPK antibodies;

FIG. 5B is a graphical representation of the staining results in FIG. 5A:

FIG. 6 shows an image of the liver tissue of MKRN14-mice in which non-alcoholic steatohepatitis (NASH) is induced (A); and the results of staining the liver tissue with Oil Red O (B):

FIG. 7 shows the results of staining the liver tissue of MKRN1-4-mice in which non-alcoholic steatohepatitis (NASH) is induced with hematoxylin & eosin (H&E) (A) and Sirius Red (B):

FIG. 8A shows the results of confirming the degree of fat accumulation after HepG2 cells are treated with an AMPK activator and PAOA:

FIG. 8B is a graphical representation of the results in FIG. 8A:

FIG. 9 shows the results of confirming the levels of MKRN1, AMPK, and pAMPK after HepG2 cells are treated with ebastine;

FIG. 10 shows the results of confirming the levels of MKRN1, AMPK, and pAMPK after HepG2 cells are treated with ebastine alone or treated with ebastine and MG132 together:

FIG. 11 shows the results of comparing the amount of MKRN1 protein in 293T cells by overexpressing MKRN1 WT, or 1-263, 264-482, DeltaRING, or an H307E mutant and treating the cells with ebastine;

FIG. 12A shows the results of confirming the ubiquitination of MKRN1 by overexpressing MKRN1 WT or an H307E mutant with ubiquitin in 293T cells and then treating the cells with ebastine;

FIG. 12B shows the results of confirming direct ubiquitination by reacting a purified recombinant MKRN1 264-482 protein with E1/E2, ATP, ubiquitin, and ebastine;

FIG. 13A shows the results of measuring the body weights of mice while feeding the mice a high-fat/high-fructose (HFHF) diet alone or a high-fat/high-fructose (HFHF) diet and ebastine together for 20 weeks;

FIG. 13B is an image of liver tissue isolated after mice are fed a high-fat/high-fructose (HFHF) diet alone or a high-fat/high-fructose (HFHF) diet and ebastine together for 20 weeks;

FIG. 13C shows the results of weighing liver tissue isolated after mice are fed a high-fat/high-fructose (HFHF) diet alone or a high-fat/high-fructose (HFHF) diet and ebastine together for 20 weeks;

FIG. 13D shows the results of subjecting liver tissue, which is isolated after mice are fed a high-fat/high-fructose (HFHF) diet alone or a high-fat/high-fructose (HFHF) diet and ebastine together for 20 weeks, to immunohistochemistry (IHC) using an MKRN1 antibody;

FIG. 13E shows the results of staining liver tissue, which is isolated after mice are fed a high-fat/high-fructose (HFHF) diet for 20 weeks, with hematoxylin & eosin (H&E);

FIG. 13F shows the results of staining liver tissue, which is isolated after mice are fed a high-fat/high-fructose (HFHF) diet for 20 weeks, with Sirius Red;

FIG. 13G shows the results of subjecting liver tissue, which is isolated after mice are fed a high-fat/high-fructose (HFHF) diet for 20 weeks, to immunohistochemistry (IHC) using an F4/80 antibody;

FIG. 14A shows the results of measuring the body weights of mice while feeding the mice a choline-deficient, L-amino acid-defined, high-fat diet (CDAHFD) alone or a CDAHFD and ebastine together for 6 weeks;

FIG. 14B is an image of liver tissue isolated after mice are fed a CDAHFD alone or a CDAHFD and ebastine together for 6 weeks;

FIG. 14C shows the results of weighing liver tissue isolated after mice are fed a CDAHFD alone or a CDAHFD and ebastine together for 6 weeks;

FIG. 14D shows the results of subjecting liver tissue, which is isolated after mice are fed a CDAHFD alone or a CDAHFD and ebastine together for 6 weeks, to immunohistochemistry (IHC) using an MKRN1 antibody;

FIG. 14E shows the results of staining liver tissue, which is isolated after mice are fed a CDAHFD alone or a CDAHFD and ebastine together for 6 weeks, with hematoxylin & eosin (H&E);

FIG. 14F shows the results of staining liver tissue, which is isolated after mice are fed a CDAHFD alone or a CDAHFD and ebastine together for 6 weeks, with Sirius Red;

FIG. 14G shows the results of subjecting liver tissue, which is isolated after mice are fed a CDAHFD alone or a CDAHFD and ebastine together for 6 weeks, to immunohistochemistry (IHC) using an F4/80 antibody;

FIG. 15A shows the results of measuring the body weights of mice fed a high-fat/high-fructose (HFHF) diet alone for 20 weeks or measuring the body weights of mice fed a high-fat/high-fructose (HFHF) diet and ebastine together after 16 weeks of feeding the high-fat/high-fructose (HFHF) diet;

FIG. 15B is an image of liver tissue isolated after mice are fed a high-fat/high-fructose (HFHF) diet alone for 20 weeks or fed a high-fat/high-fructose (HFHF) diet and ebastine together after 16 weeks of feeding the high-fat/high-fructose (HFHF) diet:

FIG. 15C shows the results of weighing liver tissue isolated after mice are fed a high-fat/high-fructose (HFHF) diet alone for 20 weeks or fed a high-fat/high-fructose (HFHF) diet and ebastine together after 16 weeks of feeding the high-fat/high-fructose (HFHF) diet:

FIG. 15D shows the results of subjecting liver tissue, which is isolated after mice are fed a high-fat/high-fructose (HFHF) diet alone for 20 weeks or fed a high-fat/high-fructose (HFHF) diet and ebastine together after 16 weeks of feeding the high-fat/high-fructose (HFHF) diet, to immunohistochemistry (IHC) using an MKRN1 antibody:

FIG. 15E shows the results of staining liver tissue, which is isolated after mice are fed a high-fat/high-fructose (HFHF) diet alone for 20 weeks or fed a high-fat/high-fructose (HFHF) diet and ebastine together after 16 weeks of feeding the high-fat/high-fructose (HFHF) diet, with hematoxylin & eosin (H&E):

FIG. 15F shows the results of staining liver tissue, which is isolated after mice are fed a high-fat/high-fructose (HFHF) diet alone for 20 weeks or fed a high-fat/high-fructose (HFHF) diet and ebastine together after 16 weeks of feeding the high-fat/high-fructose (HFHF) diet, with Sirius Red:

FIG. 15G shows the results of subjecting liver tissue, which is isolated after mice are fed a high-fat/high-fructose (HFHF) diet alone for 20 weeks or fed a high-fat/high-fructose (HFHF) diet and ebastine together after 16 weeks of feeding the high-fat/high-fructose (HFHF) diet, to immunohistochemistry (IHC) using an inflammatory marker F4/80; and

FIG. 15H shows the results of determining the expression levels of fibrosis-related genes and inflammation-related genes in liver tissue isolated after mice are fed a high-fat/high-fructose (HFHF) diet alone for 20 weeks or fed a high-fat/high-fructose (HFHF) diet and ebastine together after 16 weeks of feeding the high-fat/high-fructose (HFHF) diet, using RT-PCR.

### DETAILED DESCRIPTION OF EXEMPLARY EMBODIMENTS

Hereinafter, one or more exemplary embodiments of the present invention will be described in more detail through examples. However, it should be understood that these examples are provided to illustrate the one or more exemplary embodiments and are not intended to limit the scope of the present invention.

### Example 1: Confirmation of Effect of MKRN1 on Obesity/Diabetes

Makorin ring finger protein 1 (MKRN1) is an E3 ligase that promotes the degradation of AMP-activated protein kinase (AMPK), which activates catabolism during cellular metabolism. Accordingly, the effect of MKRN1 knockout was confirmed.

### 1-1. MKRN1^{-/-} Mice

MKRN1-null (MKRN14) mice were purchased from T. A. Gray (David Axelrod Institute, USA), and further backcrossed with C57BL/6 mice for ten generations (N10). MKRN1^{+/-} mice were crossed to obtain MKRN1-null mice and their wild-type littermates (MKRN1^{+/+}), which were randomly grouped for all experiments.

For obesity-related experiments, male and female MKRN1^{+/+} and MKRN1^{+/-} mice were fed a high-fat diet (HFD: Fat-to-calorie ratio: 60%; D12492, Research DIET Inc., USA) or a standard diet (Fat-to-calorie ratio: 17%; 38057, Cargill AgriPurina Inc.: South Korea). Dietary feeding began at 6 to 8 weeks of age in mice and continued for 16 weeks.

Adipose and liver tissues were isolated from MKRN14-mice fed a high-fat diet to confirm the level of AMPK activation. Western blot results showed that AMPK and phospho-AMPK increased in the adipose and liver tissues (FIG. 1).

Activation of AMPK promotes intracellular catabolism and consequently inhibits the accumulation of fat and the like, and phosphorylation is required for activation. Therefore, the increase in AMPK and phospho-AMPK means that the overall activation of AMPK has increased.

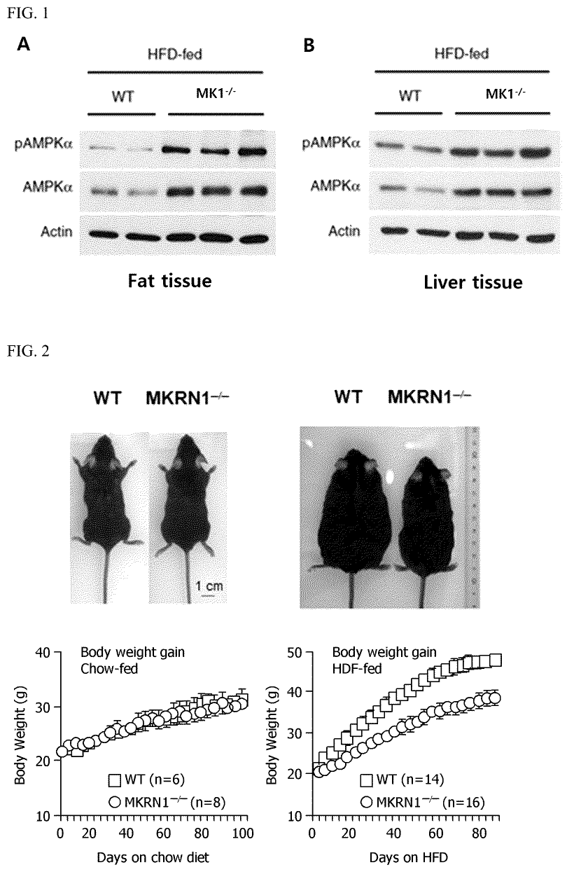

The activation of AMPK was also confirmed from the body weights of MKRN1-4-mice. That is, it can be seen that weight gain was greatly suppressed in the mice fed an HFD containing 60% fat compared to the wild-type mice (FIG. 2).

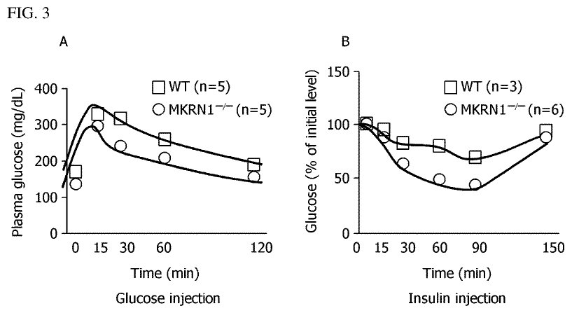

Wild-type and MKRN14-mice were fed an HFD for 16 weeks, and then fasted for 16 hours. Thereafter, oral glucose (G8769, Sigma-Aldrich) was administered at a dose of 1 g/kg for a glucose tolerance test (GTT). Blood glucose levels were analyzed at 0, 15, 30, 60, and 120 minutes after glucose injection using GlucoDr. Plus (AGM-3000, Allmedicus). As a result of the analysis, it can be seen that MKRN1^{-/-} mice had a lower level of increase in blood glucose and a faster drop in blood glucose level, compared to the control wild-type mice, even when glucose was administered (FIG. 3A).

Insulin (19278; Sigma-Aldrich) was injected intraperitoneally at a dose of 0.75 U/kg (a final volume of 125 μL) into mice that were fasted for 16 hours. Glucose levels were measured at 0, 15, 30, 60, 90, and 150 minutes after injection. As a result of the measurement, it can be seen that the blood glucose level of MKRN1^{-/-} mice recovered quickly, compared to the control wild-type mice, even when insulin was administered (FIG. 3B).

### 1-2. MKRN1 Knockout in Liver

Male wild-type C57BL/6 mice (6 weeks old) were housed in individual cages, and fed an HFD for 9 weeks. An adenovirus expressing MKRN1 shRNA was injected via the tail vein, and the mice were sacrificed after one more week of HFD feeding.

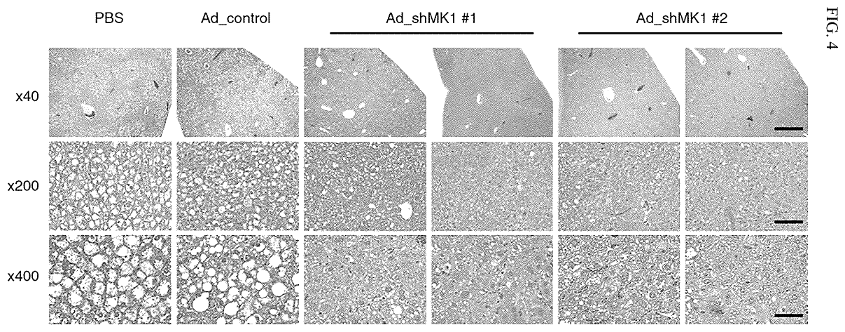

The liver tissue of the mice was isolated, and stained with hematoxylin & eosin to observe adipocytes. As a result, it can be seen that the adipocytes greatly increased due to the fat accumulation in the experimental group into which PBS or the control adenovirus was injected, whereas the adipocytes were significantly reduced in the experimental group into which the adenovirus expressing MKRN1 shRNA was injected (FIG. 4).

Based on the results in FIGS. 1 to 4, it can be seen that the obesity and diabetes can be controlled by inhibiting the function of MKRN1.

### Example 2: Confirmation of Effect of MKRN1 on Fatty Liver Disease/Steatohepatitis

Through the experiment in Example 1, it was confirmed that MKRN1 can be used as a therapeutic target for metabolic diseases such as obesity and diabetes. Accordingly, the role of MKRN1 in non-alcoholic fatty liver disease, which is a disease that progresses from obesity, was confirmed.

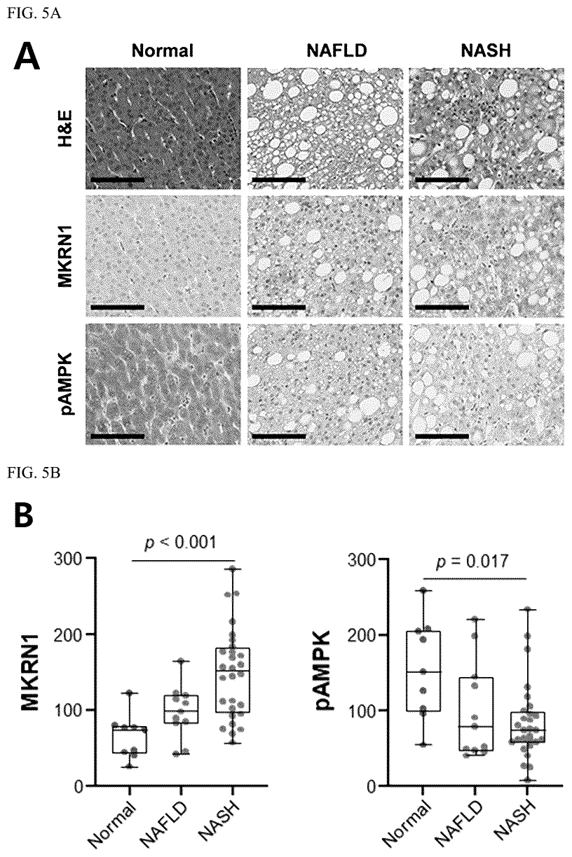

To investigate the clinical significance of MKRN1, the expression patterns of MKRN1 and AMPK were determined in the livers of patients with non-alcoholic fatty liver disease (NAFLD) and non-alcoholic steatohepatitis (NASH). As a result, it can be seen that the expression of MKRN1 significantly increased, while the expression of AMPK was suppressed (FIG. 5).

After the clinical significance of MKRN1 was confirmed, mouse experiments were carried out. NASH was induced in control and MKRN1^{-/-} mice by feeding the mice a high fat/high fructose (HFHF) diet, which is capable of inducing NASH, for 30 weeks.

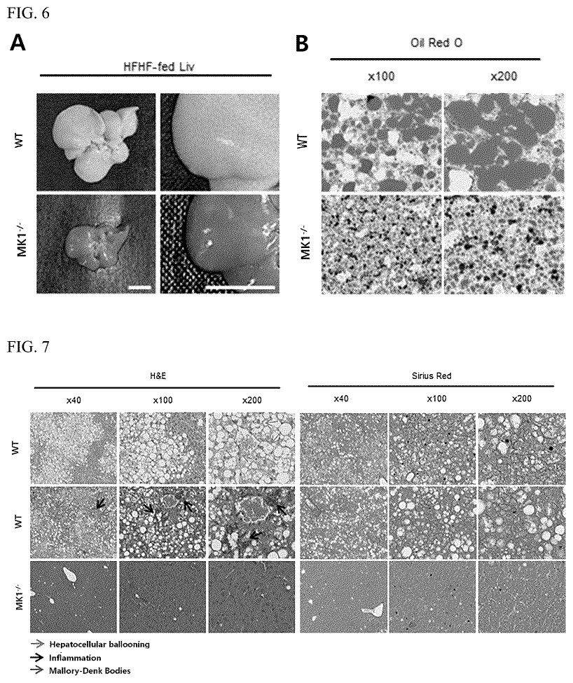

After 30 weeks, the liver was isolated, observed with the naked eye, and stained with Oil Red O to confirm the degree of fatty liver. As a result, it can be seen that the liver of the control mouse had a lot of fat accumulation even when observed with the naked eye, while the liver of the MKRN1^{-/-} mouse maintained a normal shape (FIG. 6). Based on these results, it can be seen that the MKRN1^{-/-} mice were highly resistant to NASH.

Further tissue examination of the liver showed that a great deal of cell death occurred in the liver of the control mice, but this phenomenon was not observed in the liver of MKRN1^{-/-} mice. Also, it was confirmed through Sirius Red collagen staining that severe fibrosis occurred in the liver of the control mice, and that no fibrosis phenomenon was observed in the liver of MKRN1^{-/-} mice (FIG. 7).

### Example 3: Screening of MKRN1 Inhibitor

### 3-1. Screening of Fat Accumulation-Inhibiting Substances

Because MKRN1 is an E3 ligase that is involved in the reaction of various proteins, it is difficult to screen for substances that directly target the MKRN1 protein.

Accordingly, the present inventors screened for substances that activate AMPK and identified candidate substances.

Specifically, HepG2 cells were cultured, and then treated with various types of AMPK activators. Thereafter, the cells were treated with palmitic acid and oleic acid (PAOA) at a concentration of 1 mM, and further cultured, and the degree of fat accumulation was then confirmed.

The AMPK activators used were as follows: ebastine, loratadine, desloratadine, terfenadine, fexofenadine, acrivastine, cetirizine, rupatadine, clemastine, cyproheptadien, ketotifen, mepytramine, ranitidine, cimetidine, famotidine, JNJ-5207852, and JNJ-7777120.

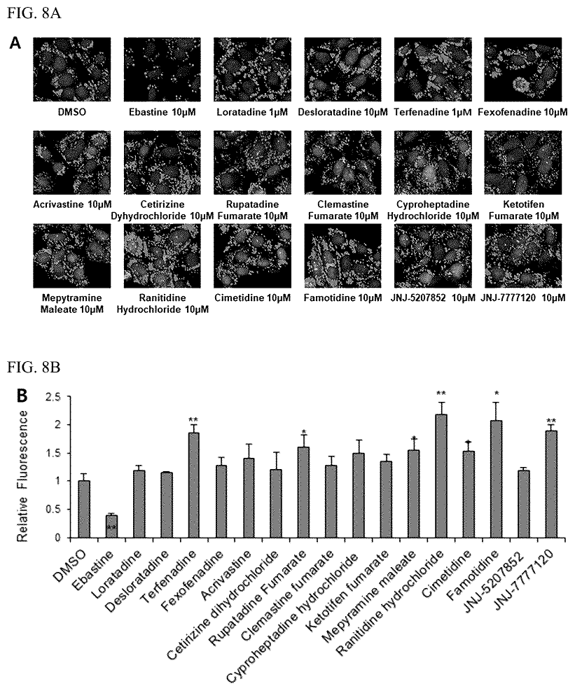

As a result, it can be seen that only ebastine was effective in suppressing fat accumulation (FIG. 8).

### 3-2. Confirmation of MKRN1 Inhibition by Ebastine

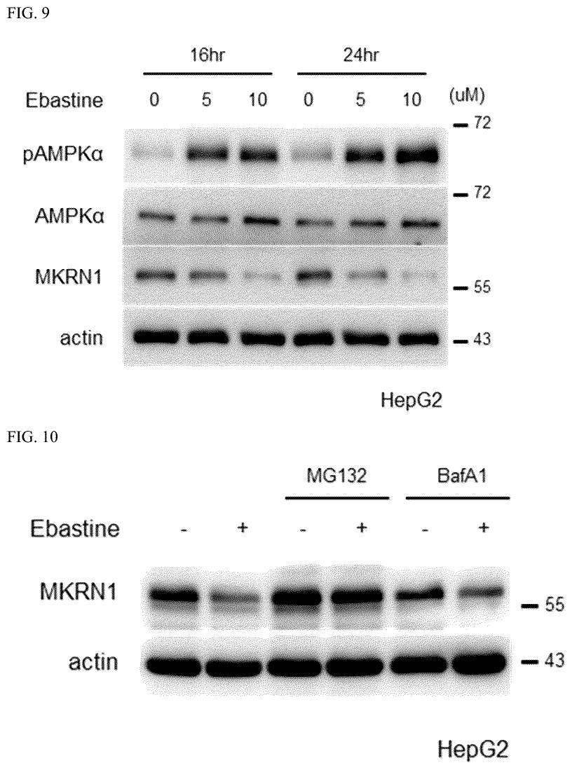

To determine whether the expression or activity of MKRN1 was suppressed by ebastine, HepG2 cells were treated with ebastine and cultured for 16 or 24 hours. After the culture was completed, the cells were recovered to measure the amount of MKRN1 protein. As a result, it can be seen that the amount of MKRN1 protein decreased and the amount of AMPK and phospholylated AMPK increased when the cells were treated with ebastine (FIG. 9).

Also, in order to confirm the mechanism by which the amount of MKRN1 protein decreases by treatment with ebastine, HepG2 cells were treated with the proteasome inhibitor MG132 along with ebastine. Then, the amount of MKRN1 protein was determined. As a result, it can be seen that the decrease in MKRN1 protein was suppressed by MG132 treatment, indicating that the decrease in MKRN1 protein by ebastine treatment was caused by the proteasome (FIG. 10).

Next, in order to confirm which portion of MKRN1 is targeted and regulated by ebastine, MKRN1 WT, and 1-263, 264-482, DeltaRING or an H307E mutant were overexpressed in 293T cells, and the 293T cells were then treated with ebastine.

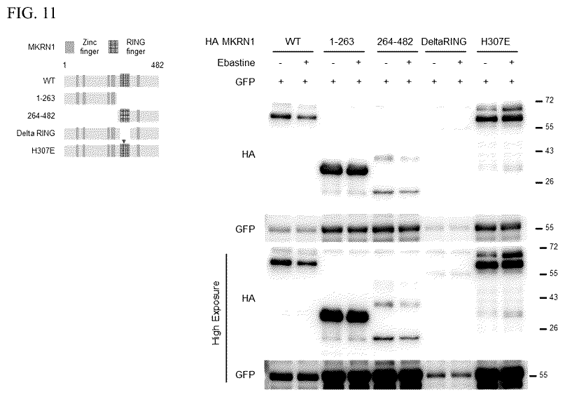

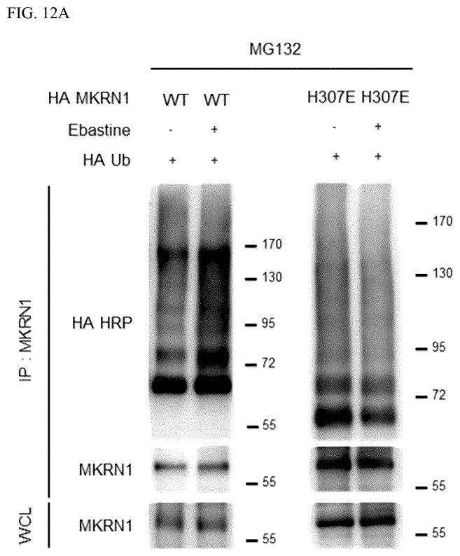

The amount of MKRN1 protein was determined. As a result, it can be seen that the amount of MKRN1 protein decreased in the MKRN1 WT and 264-482 experimental groups containing a RING finger portion of MKRN1. On the other hand, it was confirmed that ebastine did not reduce the amount of MKRN1 protein in the MKRN1 E3 ligase-deficient mutant (H307E) experimental group (FIG. 11).

Accordingly, in order to confirm whether ebastine regulates the self-ubiquitination of MKRN1, MKRN1 WT or the H307E mutant was overexpressed with ubiquitin in 293T cells, and the 293T cells were then treated with ebastine.

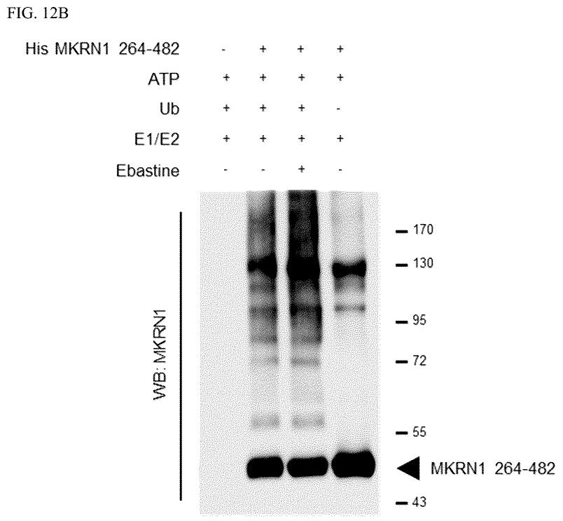

As a result, it was confirmed that ebastine induced the ubiquitination of MKRN1 in the experimental group overexpressing MKRN1 WT (FIG. 12A). The recombinant MKRN1 (264-482) protein was purified to confirm whether ebastine directly regulates the ubiquitination of MKRN1. As a result, it was also confirmed that the ubiquitination of MKRN1 (264-482) was increased by ebastine (FIG. 12B).

### Example 4: Confirmation of Anti-Obesity/Anti-Steatohepatitis Effect of Ebastine

### 4-1. Confirmation of Anti-Obesity/Anti-Steatohepatitis Effect

To confirm the anti-obesity and anti-steatohepatitis effect of ebastine, 8-week-old wild-type mice were fed an HFHF diet for 20 weeks, and ebastine was administered at concentrations of 0, 1, and 5 mg/kg after 4 weeks of feeding the HFHF diet. Body weights were measured once every 3 to 4 days for 20 weeks, and the liver was isolated at the end of the experiment. Thereafter, a histological examination was performed.

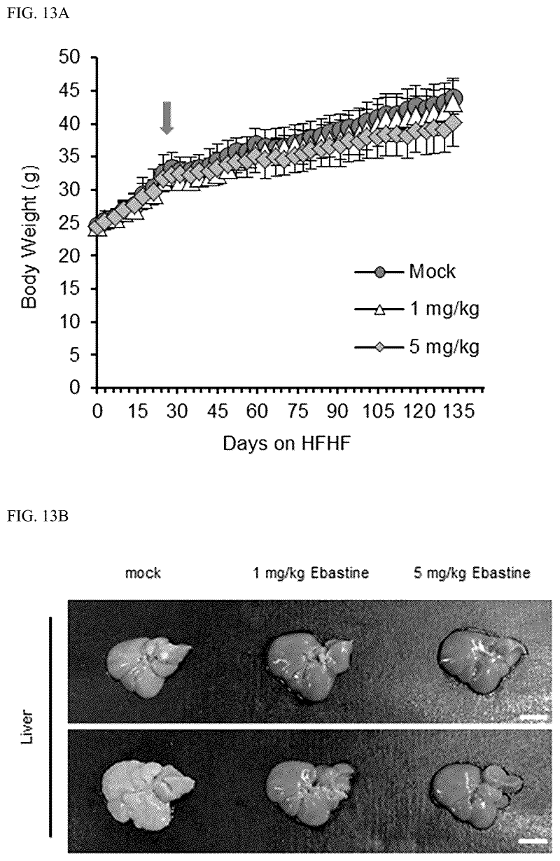

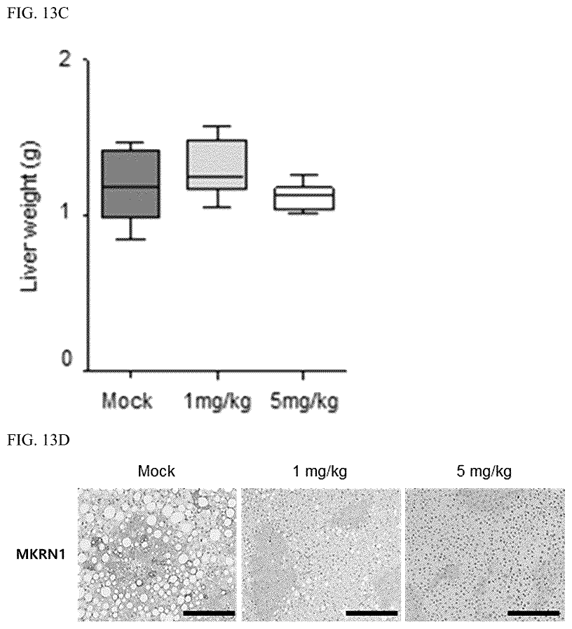

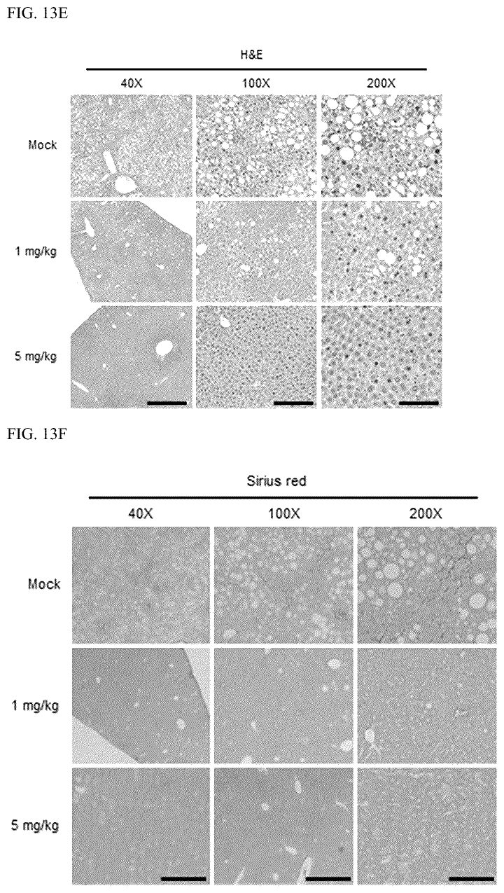

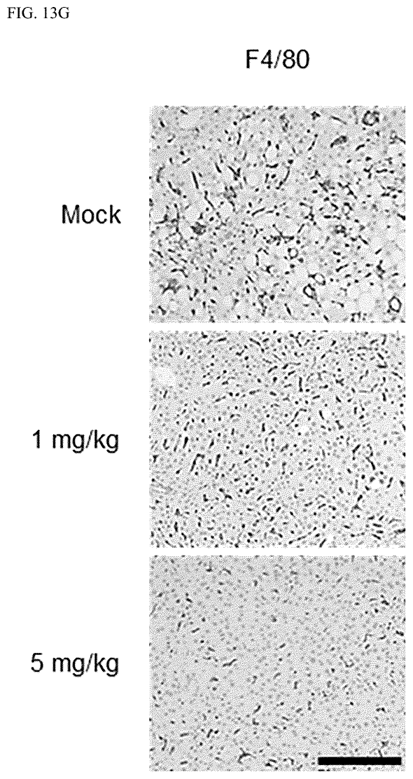

The body weights of the mice were measured. As a result, it can be seen that the weight gain of the ebastine-administered group was less compared to the control not administered ebastine (FIG. 13A). When the liver was observed with the naked eye, it can be seen that the liver of the ebastine-administered group was close to a normal shape (FIG. 13B), and the weight of the liver was lowest in the experimental group administered ebastine at a concentration of 5 mg/kg (FIG. 13C).

Also, the expression level of MKRN1 in the liver was determined through immunohistochemistry. As a result, it can be seen that the expression of MKRN1 increased in the control, whereas the level of MKRN1 expression decreased in the ebastine-administered group compared to the control (FIG. 13C).

The degree of hepatic steatosis was determined. As a result, in the ebastine-administered group compared to the control, it can be seen almost no fat accumulated in the liver (FIG. 13E), liver fibrosis did not progress (FIG. 13F), and liver inflammation was also suppressed (FIG. 13G).

It was confirmed as follows whether ebastine exhibits an anti-steatohepatitis effect even when steatohepatitis was induced in mice using various diets.

Eight-week-old wild-type mice were fed a choline-deficient, L-amino acid-defined, high-fat diet (CDAHFD) for 6 weeks, and ebastine was administered at concentrations of 0, 1, and 5 mg/kg after 2 weeks of feeding the CDAHFD. Body weights were measured once every 3 to 4 days for 6 weeks, and the liver was isolated at the end of the experiment. Thereafter, a histological examination was performed.

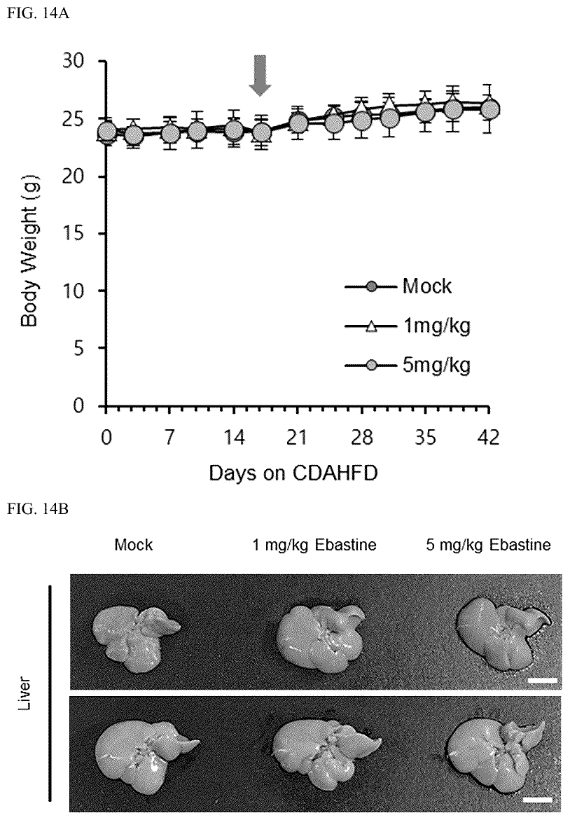

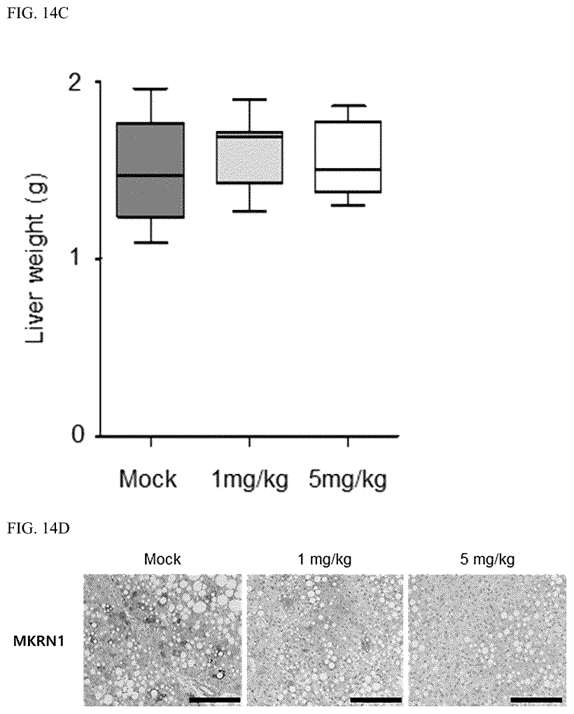

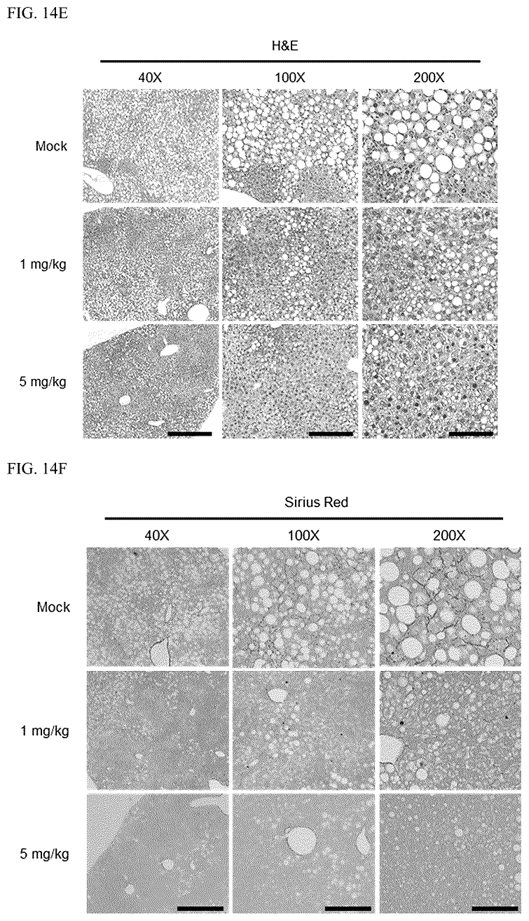

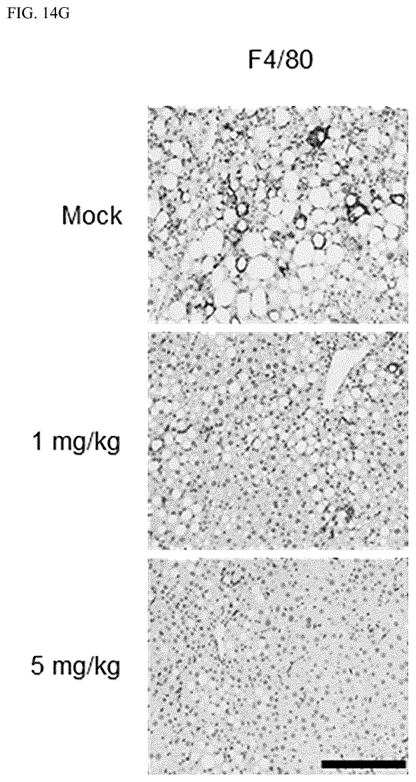

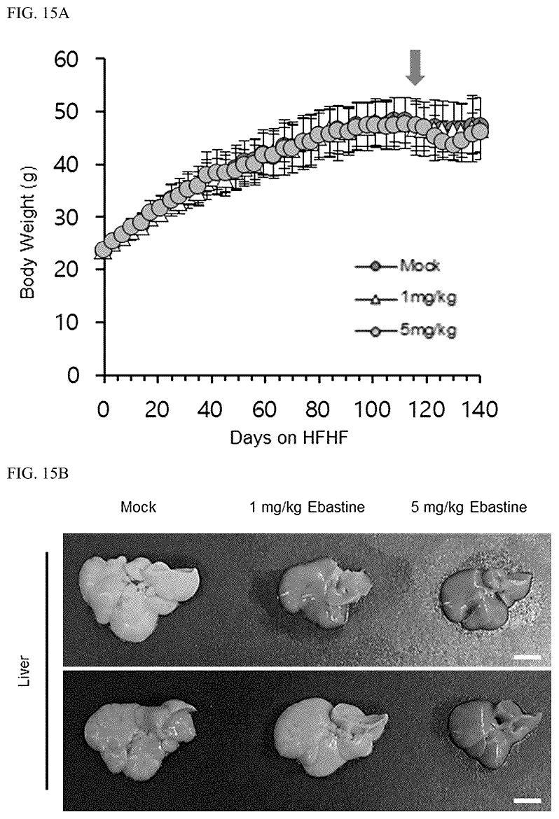

The body weights of the mice were measured. As a result, there was no significant difference in body weight between the control and the ebastine-administered group due to the characteristics of a CDAHFD, which does not induce obesity (FIG. 14A). When the liver was observed with the naked eye, the liver conditions of the control and the ebastine-administered group appeared similar (FIG. 14B). Also, it was confirmed that there was no difference in liver weight between the control and the ebastine-administered group (FIG. 14C).

To compare the difference between the control and the ebastine-administered group at a histological level, the expression level of MKRN1 in the liver was determined through immunohistochemistry. As a result, it can be seen that the expression level of MKRN1 increased in the control. On the other hand, it was confirmed that the expression level of MKRN1 decreased in the ebastine-administered group compared to the control (FIG. 14D).

Also, in the ebastine-administered group compared to the control, almost no fat accumulated in the liver (FIG. 14E), liver fibrosis did not progress (FIG. 14F), and liver inflammation was also suppressed (FIG. 14G).

### 4-2. Confirmation of Therapeutic Effect on Steatohepatitis

The therapeutic effect of ebastine on steatohepatitis was confirmed as follows.

Eight-week-old wild-type mice were fed an HFHF diet for 20 weeks, and ebastine was administered at concentrations of 0, 1, and 5 mg/kg after 16 weeks of feeding the HFHF diet. Body weights were measured once every 3 to 4 days for 20 weeks, and the liver was isolated at the end of the experiment. Thereafter, a histological examination was performed.

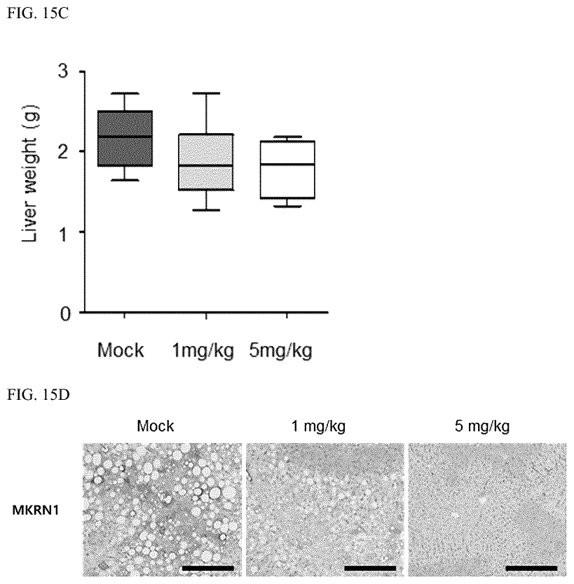

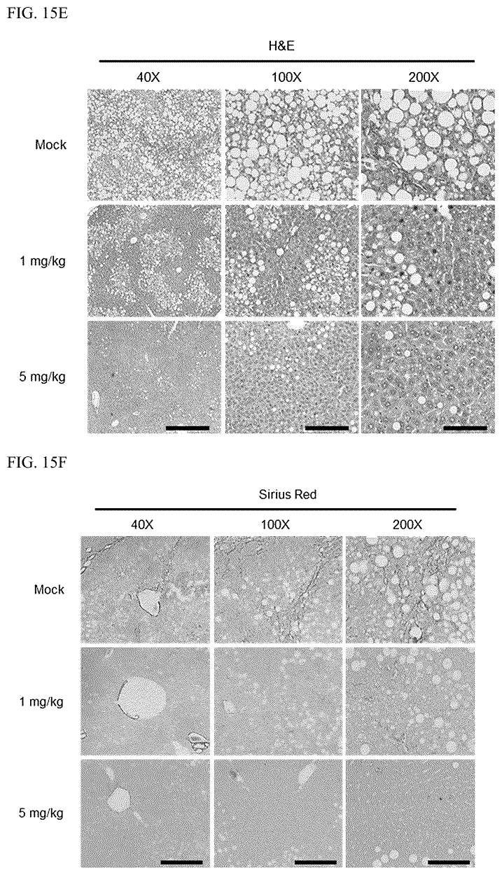

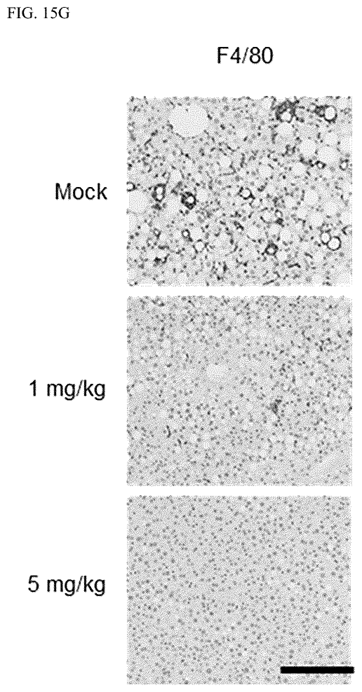

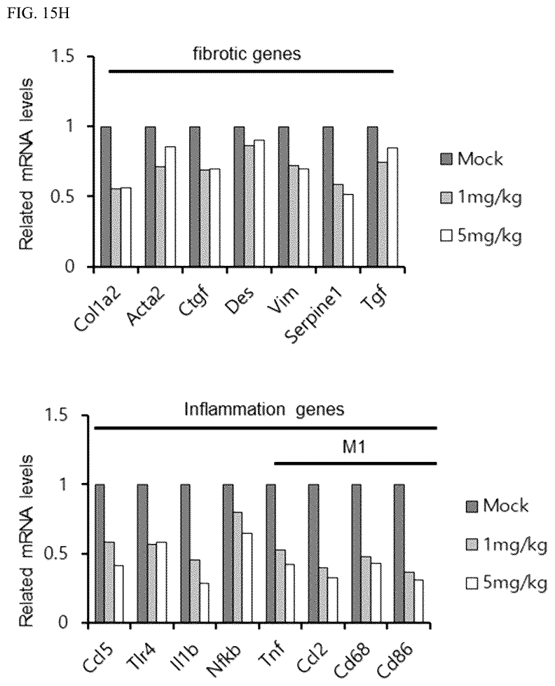

The body weights of the mice were measured. As a result, it can be seen that there was no difference in body weight change between the control not administered ebastine, and the ebastine-administered group for 4 weeks (FIG. 15A). Also, it can be seen that the liver of the ebastine-administered group was close to a normal shape when the liver was observed with the naked eye (FIG. 15B), and that the weight of the liver was lowest in the group administered 5 mg/kg of ebastine (FIG. 15C).

To compare the difference between the control and the ebastine-administered group at a histological level, the expression level of MKRN1 in the liver was determined through immunohistochemistry. As a result, it was confirmed that the expression level of MKRN1 increased in the control, whereas the expression level of MKRN1 decreased in the ebastine-administered group compared to the control (FIG. 15D).

Also, in the ebastine-administered group compared to the control, almost no fat accumulated in the liver (FIG. 15E), liver fibrosis did not progress (FIG. 15F), and liver inflammation was also suppressed (FIG. 15G).

Further, the expression levels of fibrosis- and inflammation-related genes in the liver of the mice were compared by RT-PCR. As a result, it was confirmed that the expression levels of the genes were generally suppressed in the ebastine-administered group compared to the control (FIG. 15H).

Ebastine according to one embodiment of the present invention can be used to effectively treat metabolic diseases such as obesity, diabetes, fatty liver disease, or steatohepatitis because it effectively suppresses the expression of MKRN1.

What is claimed is:

1. A method of treating fatty liver disease, comprising:
administering ebastine or a pharmaceutically acceptable salt thereof to a subject in need of treatment.

2. The method of claim 1, wherein the fatty liver disease is non-alcoholic fatty liver disease.

3. The method of claim 2, wherein the non-alcoholic fatty liver disease is non-alcoholic steatohepatitis.

4. The method of claim 1, wherein the ebastine or pharmaceutically acceptable salt thereof suppresses one or more symptoms selected from the group consisting of hepatocyte apoptosis, liver fibrosis, and fat accumulation.

5. A method of treating obesity or diabetes, comprising:
administering ebastine or a pharmaceutically acceptable salt thereof to a subject in need of treatment.

6. The method of claim 5, wherein the ebastine or pharmaceutically acceptable salt thereof provides an effect of ameliorating symptoms, such as suppressing weight gain and improving impaired glucose tolerance.

7. A composition for inhibiting makorin ring finger protein 1 (MKRN1), comprising ebastine or a pharmaceutically acceptable salt thereof as an active ingredient.
